(19) United States
(12) Patent Application Publication
(10) Pub. No.: US 2004/0126760 A1
(43) Pub. Date: Jul. 1, 2004

(54) Novel compositions and methods for carrying out multple pcr reactions on a single sample

(76) Inventor: Natalia Broude, Natick, MA (US)

Correspondence Address: Ronald I. Eisenstein, NIXON PEABODY, LLP, 100 Summer Street, Boston, MA, 02110, US

(21) Appl. No.: 10/276,390

(22) PCT Filed: May 17, 2001
(86) PCT No.: PCT/US01/15981

(51) Int. Cl. C12Q 1/68; C12P 19/34

(52) U.S. Cl. 435/6; 435/91.2

(57) ABSTRACT

The present invention provides a method for multiplex polymerase chain reaction (mpxPCR), the simultaneous amplification of different target nucleic acide sequences in a single PCR reaction. This method uses the PCR suppression effect to allow target-specific amplification with only a single target-specific primer for each target sequence. This invention further provides primers that allow simultaneous amplification of multiple DNA target sequences present in a DNA or RNA sample.

### FIELD OF THE INVENTION

The present invention is directed to novel methods and compositions for use with multiplex polymerase chain reaction (PCR). Multiplex PCR refers to the simultaneous amplification of different target nucleic acid sequences in a single PCR reaction. This method uses PCR suppression effect to obtain target-specific amplification with only a single target-specific primer for each target.

### BACKGROUND OF THE INVENTION

Polymerase chain reaction is a method whereby virtually any DNA or mRNA sequence can be selectively amplified. The method involves using paired sets of oligonucleotides of predetermined sequence that hybridize to opposite strands of the template nucleic acid, e.g., DNA, and define the limits of the sequence to be amplified. In each round of amplification, the template is duplicated. Multiple sequential rounds of DNA synthesis are catalyzed by a thermostable DNA polymerase. Each round of synthesis is typically separated by a melting and re-annealing step, allowing a given sequence to be amplified several hundred-fold in less than an hour (Saiki et al., Science 239:4-87, 1988).

The simplicity and reproducibility of these reactions has given PCR broad applicability. For example, PCR has gained widespread use for the diagnosis of inherited disorders and susceptibility to disease. Typically, the genomic region of interest is amplified from either genomic DNA or from a source of specific cDNA encoding the desired gene product. Mutations or polymorphisms are then identified by subjecting the amplified DNA to analytical techniques such as DNA sequencing, hybridization with allele specific oligonucleotides, restriction endonuclease cleavage or single-strand conformational polymorphism (SSCP) analysis.

For the analysis of small genes or genes where the mutant allele or polymorphism is well characterized, amplification of single defined regions of DNA is typically sufficient. However, when analyzing large and/or undefined genes, multiple individual PCR reactions are often required to identify critical base changes or deletions (van Orsow et al., Genomics 52: 27-36 (1998)). Thus, to streamline the analysis of large complex genes, multiplex PCR (i.e., the simultaneous amplification of different target DNA sequences in a single PCR reaction) has been proposed. A review of multiplex PCR appears in Edwards and Gibbs, PCR Methods Appl. 3: S65-75 (1994).

A significant limiting factor in being able to carry out multiplex PCR reactions is the complexity introduced by the use of multiple primers in the same reaction. To amplify N targets, typically 2N primers are required for a standard PCR reaction. One complexity associated with the presence of increasing numbers of primers is that the primer sequences can cross anneal and are therefore not available for amplifying target DNA. Five targets in a single reaction is the current maximum which can be amplified with routine ease; to amplify 20 targets requires considerable optimization of the reaction conditions.

For use in multiplex PCR, a primer should be designed so that its predicted hybridization kinetics are similar to those of the other primers used in the same multiplex reaction. While the annealing temperatures and primer concentrations may be calculated to some degree, conditions generally have to be empirically determined for each multiplex reaction. Since the possibility of non-specific priming increases with each additional primer pair, conditions must be modified as necessary as individual primer sets are added. Moreover, artifacts that result from competition for resources (e.g., depletion of primers) are augmented in multiplex PCR, since differences in the yields of unequally amplified fragments are enhanced with each cycle. Given these limitations, the development of a new diagnostic test can be very labor-intensive and costly.

Shuber (U.S. Pat. No. 5,882,856) provides chimeric primers for multiplex PCR. These chimeric primers are comprised of two sections, a 5′ end which is unrelated to the target DNA and has the property of forming hybrids with high melting temperatures; and a 3′ end which comprises a target-specific sequence. However, while these chimeric primers simplify certain aspects of primer design, they do not reduce the total number of primers (2N) required to amplify a given number of target sequences, and difficulties in designing the target-specific ends of the primers remain.

Furthermore, the results obtained with multiplex PCR are frequently complicated by artifacts of the amplification procedure. These include “false-negative” results due to reaction failure and “false-positive” results such as the amplification of spurious products, which may be caused by annealing of the primers to sequences which are related to, but distinct from, the true recognition sequences.

Chenchik et al. (U.S. Pat. Nos. 5,565,340 and 5,759,822) provides a method for decreasing artifacts generated during PCR by utilizing a PCR suppression effect (Lukyanov et al., Anal. Biochem. 229: 198-202 (1995); Siebert et al., Nucl. Acid. Res. 23: 1087-8 (1995)). This method uses novel adapters that are ligated to the end of a DNA fragment prior to PCR amplification. Upon melting and annealing, single-stranded DNA fragments having self-complementary adapters at the 5′-and 3′-ends of the strand can form suppressive “tennis racquet” shaped structures that suppress amplification of the fragments during PCR

Weighardt et al. (PCR Methods and App. 3:77, 1993) describe the use of 5′-tailed oligonucleotides for PCR However, a key feature of this amplification method involves separate annealing and primer extension reactions for each primer, which is not practical in a multiplex context.

Thus, there is a need in the art for primers and methods that allow multiplex PCR reactions to be designed and carried out without elaborate optimization steps, irrespective of the potentially divergent properties of the different primers used. Reducing the number of primers that need to be used would be desirable.

### SUMMARY OF THE INVENTION

The present invention is directed to a method for multiplex polymerase chain reaction (mpxPCR), the simultaneous amplification of different target nucleic acid sequences in a single PCR reaction. This method uses the PCR suppression effect to allow target-specific amplification with only a single target-specific primer for each target sequence. This invention further provides primers that allow simultaneous amplification of multiple DNA target sequences present in a RNA or DNA sample.

The present invention provides increased multiplexing ability with a decreased number of sequence-specific primers required.

According to the present invention, nucleic acid templates to be amplified in a multiplex PCR reaction are first prepared as tennis racquet structures to allow the PCR suppression effect. For example, the DNA sample in a single reaction mixture is contacted with a set of oligonucleotide primers, wherein the set of oligonucleotide primers is comprised of one universal adapter primer and one target-specific primer for each target sequence. Thus, the total number of primers required to amplify a given number of targets in a single PCR reaction is N+1, not 2N, wherein N is the number of targets to be amplified.

Multiple cycles of melting, reannealing, and synthesis (i.e., a PCR reaction) are thereafter performed with the above mentioned sample and the oligonucleotide primers. Amplified target sequences may then be detected by any method, including, for example, gel electrophoresis followed by hybridization, in which the presence or absence of an amplification product is diagnostic of the presence or absence of the target sequence in, for example, the original DNA sample. In other embodiments, the amplification product is detected with allele-specific oligonucleotides, restriction endonuclease cleavage, or single-strand conformational polymorphism (SSCP) analysis.

In another aspect, the invention encompasses methods for high-throughput genetic screening. The method allows the rapid and simultaneous detection of multiple defined target DNA sequences in DNA samples obtained from a multiplicity of individuals. It is carried out by simultaneously amplifying many different target sequences from a large number of desired samples, such as patient DNA samples, using oligonucleotide primers as above.

In yet another aspect, the present invention provides single-stranded oligonucleotide DNA primers for amplification of a target DNA sequence in a multiplex polymerase chain reaction. The primers to amplify each target are comprised of a universal adapter-primer and a target-specific primer.

The methods and compositions of the present invention can be applied to the diagnosis of genetic and infectious diseases, gender determination, genetic linkage analysis, and forensic studies.

### BRIEF DESCRIPTION OF THE DRAWINGS

FIG. 1 provides a schematic outlining the PCR suppression effect which allows the selective amplification of targeted sequences from genomic DNA using one target-specific primer and one universal adapter-primer. DNA is digested with a restriction enzyme, and the ends of the resultant DNA fragments are tagged by ligation with complementary adapter sequences. After filling in the ends of the adapter sequences all fragments are entirely double-stranded. During PCR, when two strands are separated, each single-stranded fragment has self-complementary ends which favors the formation of stem-loop structures due to intramolecular base pairing of the adapter sequences since the adapters contain long G/C rich oligonucleotides. PCR amplification using a universal adapter-primer (which is complementary to the adapter sequence) is inhibited by the double-stranded nature of the stem portion of the template. However, amplification with a target-specific primer is efficient if the target sequence is present within the single-stranded loop region. The presence of the universal adapter-primer in the reaction subsequently allows the product of the first amplification reaction to be further amplified.

FIGS. 2A-C show the results of PS-based mpxPCR targeted at several anonymous sequences in chromosome 7 DNA. 5 ng of RsaI-digested human genomic DNA with ligated adapter was amplified in a 25 μl reaction containing 1× PCR buffer (PE), 2.5 mM MgCl₂, 250 μM dNTP, 2.5 U of thermostable DNA polymerase (a (1:1:1) mixture of Taq, AmpliTaq and AmpliTaq Gold DNA polymerases) and 5 pmole of each primer. The fluorescently labeled A-primer was TGTAGCGTGAAGACGACAGAA (SEQ ID NO:1), which corresponded to the 5′ outermost part of the ligated adapter. For the T-primers see Table 1. FIG. 2A depicts RsaI restriction maps of two fragments from human chromosome 7 with the PCR primers. FIG. 2B is a gel showing resolution of mpxPCR products using 2% agarose gel electrophoresis. 1: PCR with the T-primers 1+2; 2: PCR with the T-primers 3+4; 3: PCR with the T-primers 1+2+3+4 (see Table 1), M: 100 bp size marker. FIG. 2C shows resolution of the 4-plex (top, primers 1-4, Table 1) and 5-plex (bottom, primers 1-5, Table 1) PCR products using 6% PAGE and an ALF sequencer. The numbers above the peaks show the amplicon lengths. The 1 kb-long PCR amplicon is beyond the displayed window.

FIGS. 3A-D show the results of 14-fold mpxPCR. In FIG. 3A, 5-plex PCR was performed with primers 1-5 (Table 1); in FIG. 3B, 4-plex PCR was performed with primers 2, 3, 5 and 8 (Table 2); FIG. 3C shows 5-plex PCR with primers 1, 4, 6, 7, and 9 (Table 2): and FIG. 3D shows the results of 14-plex PCR with primers 1-5 (Table 1) plus primers 1-9 (Table 2). Primers 2, 3, and 5 (Table 2) were designed for wild type sequences. Other conditions were as in FIG. 2. The 450 bp fragment (aldolase B gene, primer 4, Table 2) appeared as a double peak, as it also did in a uniplex PCR (data not shown). The 1 kb-long fragment (product generated by the primer 5, Table 1) is beyond the displayed window. The fragment's lengths (bp) are indicated above the peaks. The 200 bp-long IL2 amplicon is marked by an arrow.

FIG. 4 shows that different DNA polymerases display different specificity in mpxPCR. 4-plex PCR used primers 1, 2, 3, and 4, Table 1. 2.5 U of thermostable DNA polymerase or 2.5 U of a mixture of two DNA polymerases was used in PCR. Other conditions were as in FIG. 2. PD: primer-dimers.

FIGS. 5A-C shows the results of allele-specific mpxPCR. FIG. 5A shows the results of uniplex reactions with wild type (N) and mutated (M) primers targeting the interleukin-2 gene (IL2); neurofibromatosis (NFM) gene; and alpha-2-macroglobulin (MG) gene. In FIG. 5B, 4-plex PCR was performed with an equimolar mixture of wild type (N) or mutant (M) primers. The fourth target in both reactions was a fragment of the integrin B₂ subunit gene (primer 8, Table 2). The PCR products were resolved on a 2% agarose gel. FIG. 5C shows a window showing the absence of two targets (MG and IL2, marked by arrows) amplified with the mutant primers in a 14-plex PCR. PCR products were resolved using 6% PAGE in an ALF sequencing instrument.

FIGS. 6A-B show genotyping of cystic fibrosis (CF) human DNA samples. FIG. 6A shows uniplex reactions with primers targeting wild type (1), ΔF508 CFTR-homozygous (2), and ΔF508 CFTR-heterozygous (3) DNA. The PCR products were analyzed on a 2% agarose gel. FIG. 6B shows genotyping of the same DNA samples in a 4-plex PCR. In this reaction CFTR wild type (n) and CFTR mutant (m) primers (primers 10 and 11, respectively, Table 2) were used in a mixture with primers 1, 2 and 4 (Table 1) (see the Examples for the complete details).

### DETAILED DESCRIPTION OF THE INVENTION

The present invention is directed to a method for multiplex polymerase chain reaction (PCR), the simultaneous amplification of different target nucleic acid sequences in a single PCR reaction. This invention further provides primers that allow simultaneous amplification of multiple target sequences present in a nucleic acid sample.

According to the invention, the nucleic sample in a single reaction mixture is contacted with a set of oligonucleotide primers, wherein the set of oligonucleotide primers is comprised of one universal adapter-primer and one target-specific primer for each target sequence. Thus, the total number of primers required to amplify a given number of targets in a single PCR reaction is N+1, wherein N is the number of targets to be amplified.

The method of the present invention is comprised of the following steps. First, the nucleic acid of interest (i.e. containing the putative target sequences) is cleaved with a restriction enzyme, generating a population of double-stranded DNA fragments. Second, adapter sequences are ligated to both ends of each DNA fragment, such that following denaturation, the ends of the resultant single strands (or the ligated adapters) are complementary and anneal to form a double-stranded stem of the stem loop structure. The region of the genomic DNA forms single-stranded loop. Third, a set of oligonucleotide primers is added, including one universal adapter-primer and one target-specific primer for each target sequence. Fourth, the PCR reaction is performed. The universal adapter-primer alone cannot initiate DNA amplification, as its binding to its complementary sequence is highly inefficient due to the double-stranded nature of the stem region. The target-specific primer however is able to bind its complement if the target sequence is present within the loop region, leading to amplification of the target which continues into the stem region (albeit with reduced efficiency). Finally, in subsequent amplification cycles the universal adapter-primer (which recognizes the stem region) provides the second primer for a traditional PCR reaction, and the segment is amplified exponentially.

“Amplification” of DNA as used herein denotes the use of polymerase chain reaction (PCR) to increase the concentration of a particular DNA sequence within a mixture of DNA sequences.

“Multiplex PCR” (mpx PCR) as used herein refers to the simultaneous amplification of multiple nucleic acid targets in a single polymerase chain reaction (PCR) mixture.

“High-throughput” denotes the ability to simultaneously process and screen a large number of nucleic acid samples (e.g. in excess of 100 genomic DNAs) in a rapid and economical manner, as well as to simultaneously screen large numbers of different genetic loci within a single nucleic acid sample.

To form the stem-loop structure of the present invention, the DNA sample to be analyzed is first cleaved with a restriction enzyme. Any restriction enzyme which allows ligation to the adapter sequences may be used. Adapter sequences are ligated to both ends of each DNA fragment. Any overhanging single-stranded ends will be filled in during the first seconds of the first PCR cycle. Following denaturation of the DNA, the ends of the resultant single strands are complementary and anneal to form a stem-loop structure. The adapter sequences anneal to form the double-stranded loop, and the region of the genomic DNA fragment forms the single-stranded loop. This stem-loop structure is the template used for the multiplex PCR reaction.

The adapters of the present invention are oligonucleotides which may be partially double-stranded. Preferably, the adapters are at least partially double-stranded to aid in ligation of the adapter to the nucleic acid fragment. The adapters can be attached to the ends of DNA fragments using a variety of techniques that are well known in the art, including DNA ligase-mediated ligation of the adapters to sticky- or blunt-ended DNA and T4 RNA ligase-mediated ligation of a single-stranded adapter to single-stranded DNA. As used herein, the term “attach,” when used in the context of attaching the adapter to a nucleic acid fragment, refers to bringing the adapter into covalent association with the nucleic acid fragment regardless of the manner or method by which the association is achieved.

Alternatively, DNA fragments can be cloned into plasmid vectors that have the adapters of the present invention inserted in the appropriate orientation upstream and downstream of a cloning site in the vector. In this case, the adapters per se consist of a single vector sequence in the plasmid that duplicates the adapter sequence upstream and downstream from a cloning site. The vector can include any plasmid sequence that is necessary for maintenance of the recombinant DNA in a host cell.

The adapters of the present invention are comprised of nucleic acid sequences typically not found in the population of nucleic acid templates. When the sequence of the nucleic acid templates is known (e.g. genomic DNA of certain organisms), the lack of homology between the adapter sequence and the nucleic acid template(s) may be determined using sequence comparison analysis programs well known in the art (e.g. BLAST). Alternatively, such lack of homology can be determined empirically, for example by nucleic acid hybridization techniques such as Southern blotting. Preferably, there is less than 35% identity (homology) between the adapter sequence and the template, more preferably less than 30% identity, still more preferably less than 25% identity. The sequence analysis programs used to determine homology are run at the default setting.

Preferably, the adapter sequence of the present invention includes a specific recognition sequence for a restriction enzyme. Preferably, the sequence is for a type IIs restriction enzyme near its 3′ end. For example, a HgaI site.

Several types of adapter structures are contemplated for use with the present invention. Two types of adapters are referred to herein as “Type 1” and “Type 2” adapter structures. Using the teachings contained herein, the skilled artisan could readily construct other adapters that have different sequences from those adapters exemplified herein, including variants of the subject adapters, that would be operable with the present invention. Any polynucleotide sequence that comprises a primer binding portion and an effective suppressor sequence portion and which when associated with a DNA or RNA fragment can form a suppressive stem-loop structure during PCR as described herein is contemplated by the subject invention. Such adapters are within the scope of the present invention.

The Type 1 adapter structure typically has a length of about 40-50, more preferably 42-50 nucleotides. However, the adapter length can vary from as few as about 25 nucleotides, up to 80 or more nucleotides. Generally, the Type 1 adapter does not contain any homopolymer sequence. The Type I adapter typically has a high GC content. A high GC content means that at least 40% of the base pairs are G or C, more preferably at least 45% of the base pairs, still more preferably at least 50%. The Type 1 adapter is typically at least partially double-stranded and generally comprises one long oligomer and one short oligomer, resulting in a 5′ overhang at one end of the adapter. The length of the shorter oligomer is not critical to the function of the PCR suppression method of the present invention, and can be shorter or equal to the length of the longer oligomer.

Examples of Type I adapter sequences include the following:

| | |
|---|---|
| 5′TGTAGCGTGAAGACGACAGAA AGGGCGTGGT | (SEQ ID NO: 22) |
| GCGGACGCGGG3′ | |
| 3′CGCCTGCGCCC5′ | (SEQ ID NO: 23) |
| 5′NGNNGCGNGNNGNCGNCNGNN NGGGCGNGGN | (SEQ ID NO: 24) |
| GCGGNCGCGGGN3′ | |
| 3′CGCCNGCGCCCN5′ | (SEQ ID NO: 25) |
| 5′TSTASSSTSAASASSASASAA ASSSSSTSST | (SEQ ID NO: 26) |
| SSSSASSSSSST3′ | |
| 3′SSSSTSSSSSSA5′ | (SEQ ID NO: 27) |

The design of the Type 1 adapter allows it to be ligated to any blunt-ended DNA fragment using T4 DNA ligase. Adapters having “sticky ends” that are compatible with certain restriction sites can also be used to attach the adapter to DNA that has been digested with appropriate restriction endonucleases. In most instances, only the upper (and typically longer) oligomer of the adapter can be ligated to DNA. The lower (and typically shorter) oligomer usually is not ligated because it lacks the requisite 5′-phosphate group. However, the lower oligomer portion of the adapter does increase the efficiency of ligation of the adapter to double-stranded DNA (dsDNA). In those instances where it is desirable to do so, it is possible to modify the shorter oligomer so that it can be ligated to the DNA fragment. Typically, these modifications include adding a 5′-phosphate for more efficient ligation.

The efficiency of suppression can be regulated through varying the length and GC content (which in turn determines the melting temperature of the dsDNA) of the suppressor portion of the adapter.

The Type 2 adapter structure is similar to the Type 1 structure but contains a homopolymer sequence in the suppressor portion of the adapter. Typically, the Type 2 adapter is incorporated into DNA fragments that have been tailed with oligo (dA) using terminal deoxynucleotidyl transferase, followed by PCR using an appropriate primer. In this case, the primer becomes incorporated into the DNA as an adapter. The PCR product can be subsequently treated with exonuclease III to remove the lower strand of the adapter.

The skilled artisan will readily recognize that certain of the primers of the subject invention can also be used as adapters. The use of primers as adapters in the present method, and vice versa, is contemplated by the present invention.

Preferably, the adapter should not contain any sequences that can result in the formation of “hairpins” or other secondary structures which can prevent adapter ligation and intramolecular annealing. As would be readily apparent to a person skilled in the art, the universal adapter primer binding portion of the adapter can be complementary with a PCR primer capable of priming for PCR amplification of a target DNA. The universal adapter primers of the present invention comprise a polynucleotide sequence that is complementary to a portion of the polynucleotide sequence of a suppression adapter of the invention.

The adapters and primers used in the subject invention can be readily prepared by the skilled artisan using a variety of techniques and procedures. For example, adapters and primers can be synthesized using a DNA or RNA synthesizer. In addition, adapters and primers may be obtained from a biological source, such as through a restriction enzyme digestion of isolated DNA. Preferably, the primers are single-stranded.

The present invention has an increased multiplexing ability due to the decreased number of sequence-specific primers required.

As used herein, the term “primer” has the conventional meaning associated with it in standard PCR procedures, i.e., an oligonucleotide that can hybridize to a polynucleotide template and act as a point of initiation for the synthesis of a primer extension product that is complementary to the template strand.

The universal adapter-primers of the present invention comprise a polynucleotide sequence that is complementary to a portion of the polynucleotide sequence of a suppression adapter of the invention.

Preferably, the universal adapter primer of the present invention has exact complementarity with a portion of the adapter sequence. However, primers used in the present invention can have less than exact complementarity with the primer binding sequence of the adapter as long as the primer can hybridize sufficiently with the adapter sequence so as to be extendible by a DNA polymerase.

The target-specific primers of the present invention comprise a polynucleotide sequence that is complementary to a portion of the polynucleotide sequence of the target sequence found within the loop region of the stem-loop structure.

Preferably, the target-specific primer of the present invention is completely complimentary to the target sequence. However, primers used in the present invention can have less than exact complementarity with the primer binding sequence of the target sequence as long as the primer can hybridize sufficiently with the target sequence so as to be extendible by a DNA polymerase.

For use in a given multiplex PCR reaction, the universal adapter and the target-specific primer sequences are typically analyzed as a group to evaluate the potential for fortuitous dimer formation between different primers. This evaluation may be achieved using commercially available computer programs for sequence analysis, such as Gene Runner, Hastings Software Inc. Other variables, such as the preferred concentrations of Mg⁺², dNTPs, polymerase, and primers, are optimized using methods well-known in the art (Edwards et al., PCR Methods and Applications 3:565 (1994)).

Any nucleic acid sample may be used in practicing the present invention, including without limitation eukaryotic, prokaryotic and viral DNA or RNA. In a preferred embodiment, the target nucleic acid represents a sample of genomic DNA isolated from a patient. This DNA may be obtained from any cell source or body fluid. Non-limiting examples of cell sources available in clinical practice include blood cells, buccal cells, cervicovaginal cells, epithelial cells from urine, fetal cells, or any cells present in tissue obtained by biopsy. Body fluids include blood, urine, cerebrospinal fluid, semen and tissue exudates at the site of infection or inflammation. DNA is extracted from the cell source or body fluid using any of the numerous methods that are standard in the art. It will be understood that the particular method used to extract DNA will depend on the nature of the source. The preferred amount of DNA to be extracted for use in the present invention is at least 5 pg (corresponding to about 1 cell equivalent of a genome size of 4×10⁹ base pairs).

The present method can be used with polynucleotides comprising either full-length RNA or DNA, or their fragments. The RNA or DNA can be either double-stranded or single-stranded, and can be in a purified or unpurified form. Preferably, the polynucleotides are comprised of DNA. The DNA fragments used in the present invention can be obtained from DNA by random shearing of the DNA, by digestion of DNA or cDNA with restriction endonucleases, or by amplification of DNA fractions from DNA using arbitrary or sequence-specific PCR primers. The present invention can also be used with full-size cDNA polynucleotide sequences, such as can be obtained by reverse transcription of RNA. The DNA can be obtained from a variety of sources, including both natural and synthetic sources. The DNA can be from any natural source including viruses, bacteria, yeast, plants, insects and animals. The DNA can also be prepared from any RNA source.

In practicing the present invention, a nucleic acid sample is contacted with pairs of oligonucleotide primers under conditions suitable for polymerase chain reaction. Standard PCR reaction conditions may be used, e.g., 1.5 mM MgCl.sub.2, 50 mM KCl, 10 mM Tris-HCl, pH 8.3, 200 μM deoxynucleotide triphosphates (dNTPs), and 25-100 U/ml Taq polymerase (Perkin-Elmer, Norwalk, Conn.).

The concentration of each primer in the reaction mixture can range from about 0.05 to about 4 μM. Each target primer is evaluated by performing single PCR reactions using each primer pair (a universal adapter-primer and a target-specific primer) individually. Similarly, each primer pair is evaluated independently to confirm that all primer pairs to be included in a single multiplex PCR reaction generate a product of the expected size. As the number of targets in a single reaction increases, certain targets may not be amplified as efficiently as other targets. The concentration of the primers for such underrepresented targets may be increased to increase their yield. For example, when multiplying 15 or more targets; more preferably, when multiplying 30 or more targets.

Multiplex PCR reactions are carried out using manual or automatic thermal cycling. Any commercially available thermal cycler may be used, such as, e.g., Perkin-Elmer 9600 cycler.

The present invention is preferably used with at least 5 targets, more preferably at least 10 targets, still more preferably with at least 14 targets, even more preferably with at least 20 targets, yet more preferably with at least 30 targets, still more preferably with at least 50 targets, and even more preferably with at least 100 targets.

A variety of DNA polymerases can be used during PCR with the present invention. Preferably, the polymerase is a thermostable DNA polymerase such as may be obtained from a variety of bacterial species, including Thermus aquaticus (Taq), Thermus thermophilus (Tth), Thermus filiformis, Thermus flavus, Thermococcus literalis, and Pyrococcus furiosus (Pfu). Many of these polymerases may be isolated from the bacterium itself or obtained commercially. Polymerases to be used with the present invention can also be obtained from cells which express high levels of the cloned genes encoding the polymerase. Preferably, a combination of several thermostable polymerases can be used.

The PCR conditions used to amplify the targets are standard PCR conditions, as described below in Protocol B. Typical conditions use 35-40 cycles, with each cycle comprising a denaturing step (e.g. 10 seconds at 94° C.), an annealing step (e.g. 15 sec at 68° C.), and an extension step (e.g. 1 minute at 72° C.). As the number of targets in a single reaction increases, the length of the extension time may be increased. For example, when amplifying 30 or more targets, the extension time may be three times as longer than when amplifying 10-15 targets (e.g. 3 minutes instead of 1 minute).

Finally, the reaction products are analyzed using any of several methods that are well-known in the art. Preferably, agarose gel electrophoresis is used to rapidly resolve and identify each of the amplified sequences. In a multiplex reaction, different amplified sequences are preferably of distinct sizes and thus can be resolved in a single gel. In one embodiment, the reaction mixture is treated with one or more restriction endonucleases prior to electrophoresis. Alternative methods of product analysis include without limitation dot-blot hybridization with allele-specific oligonucleotides and SSCP.

The present invention further concerns kits which contain, in separate packaging or compartments, the reagents such as adapters and primers required for practicing the multiplex PCR method of the subject invention. Such kits may optionally include the reagents required for performing PCR reactions, such as DNA polymerase, DNA polymerase cofactors, and deoxyribonucleotide-5′-triphosphates. Optionally, the kit may also include various polynucleotide molecules, DNA or RNA ligases, restriction endonucleases, reverse transcriptases, terminal transferases, various buffers and reagents, and antibodies that inhibit DNA polymerase activity. The kits may also include reagents necessary for performing positive and negative control reactions. Optimal amounts of reagents to be used in a given reaction can be readily determined by the skilled artisan having the benefit of the current disclosure.

The multiplex PCR methods of the subject invention can be used in a wide variety of procedures. Several of these procedures are discussed herein. Other procedures would become apparent to one skilled in the art having the benefit of this disclosure.

The multiplex PCR method of the present invention can be used to simultaneously amplify difference disease-related sequences under identical conditions.

The subject invention can also be used with long distance (LD) PCR technology (Barnes, 1994; Cheng et al., 1994). LD PCR, which uses a combination of thermostable DNA polymerases, produces much longer PCR products with increased fidelity to the original template as compared to conventional PCR performed using Taq DNA polymerase alone. The method of the present invention can also be used in conjunction with antibodies that bind to DNA polymerase and thereby inhibit polymerase function (Kellogg et al., 1994). These antibodies reversibly bind to DNA polymerase in a temperature-specific manner, and thereby increase the specificity of a PCR reaction by inhibiting the formation of non-specific amplification products prior to initiation of PCR amplification

### EXAMPLES

In the present invention the ability to simultaneously amplify multiple DNA targets using PCR suppression was tested for several unique targets by using a mixture of gene-specific primers. There are several issues which need to be addressed to optimize the PS-based PCR for multiplex amplification. To perform PS-based PCR, genomic DNA should be digested with an appropriate restriction enzyme and ligated with PS-adapters. That means that the design of the gene-specific primers will depend not only on the location of the tentative target, but also on the availability of the convenient restriction site/s in proximity to the target. One problem, which is inherent to all PCR-based methods with multiple targeting, is biased amplification of certain templates, while others are lost during multiple PCR cycles. These examples look at such issues.

Samples

Non-phosphorylated oligonucleotides, unlabeled or fluorescently labeled, were purchased as custom synthesis products from Integrated DNA Technologies, Inc. (Coralville, Iowa). DNA from peripheral blood lymphocytes from anonymous donors was isolated using a Qiagen Blood kit according to the manufacturer's protocol (Qiagen, Chatsworth, Calif.). DNA samples from cystic fibrosis affected individuals were kindly provided by Drs. R. Nelson and B. Allitto (Genzyme Genetics, Framingham, Mass.). AmpliTaq and AmpliTaq Gold DNA polymerases were from Applied Biosystems (Foster City, Calif.), KlenTaq DNA polymerase from Ab peptides (St. Louis, Mo.) and Taq DNA polymerase from Amersham Pharmacia Biotech.

Preparation of adapter-ligated DNA was performed as described (18). Human genomic DNA was digested with RsaI restriction enzyme (New England Biolabs, Beverly, Mass.) and ligated with adapters consisting of two annealed oligonucleotides: TGTAGCGTGAAGACGACAGAAAGGGCGTGGTGCGGACGCGGG (SEQ ID NO:22) and CCCGCGTCCGC (SEQ ID NO:23). The complementary oligonucleotides were of different lengths to insure the right polarity of ligation to blunt-ended genomic fragments. The recessed ends of the ligated DNA fragments were automatically filled in during the first round of subsequent PCR in the presence of dNTPs and DNA polymerase.

DNA Amplification

Adapter-ligated DNA (2-5 ng) was amplified by PCR in a 25 μl reaction volume containing 1× PCR buffer (depending on the DNA polymerase), 2.5 mM MgCl₂, 250 μM of each dNTP, 5-10 pmol of each primer and 2.5 U of thermostable DNA polymerase. The PCR mixtures containing all components but primers were denatured at 95° C. for 3-10 min; the primer mixture (5-10 pmol of each) was added at 95° C.; and 38 cycles of PCR (94° C., 10 sec, 68° C., 15 sec and 72° C., 1 min) were performed. The A-primer common for all targets was fluorescein-labeled, and the PCR products were analyzed by electrophoresis on a 2% agarose gel and by 6% denaturing PAGE.

To perform genotyping of cystic fibrosis (CF) DNAs in a mpxPCR, DNA samples were pre-amplified in a 15 cycle PCR with the nested cystic fibrosis transmembrane regulator (CFTR) primer 12 (Table 2), primers 1, 2, 4 (Table 1), and the A-primer, TGTAGCGTGAAGACGACAGAA (SEQ ID NO:1). Then 1 μl of 500-fold diluted PCR product was re-amplified in a 38 cycle PCR with a mixture of 4 T-primers (including CFTR normal or mutant primer, primers 10 or 11, Table 2) and the A-primer, GAAAGGGCGTGGTGCGGACGCGG (SEQ ID NO:28), using the same PCR conditions as described above.

Display and Analysis of PCR Products

PCR products (2-3 μl) were denatured for 3 min at 90° C. in a stop solution (Amersham Pharmacia Biotech), and analyzed using a 6% denaturing PAGE and an ALF sequencing instrument (Amersham Pharmacia Biotech) as described (17, 18).

Sequencing of CFTR Fragments

CFTR DNA fragments were amplified from several DNAs using direct fluorescein-labeled GGGAGAACTGGAGCCTTCAGAG (SEQ ID NO:29) and reverse GGGTAGTGTGAAGGGTTCATATGC (SEQ ID NO:30) primers. 5-10 fmol of the PCR product was sequenced using a fmol DNA sequencing kit (Promega, Madison, Wis.) according to the manufacturer's protocol and the products were resolved by 6% PAGE in an ALF sequencing instrument (Amersham Pharmacia Biotech).

General Considerations

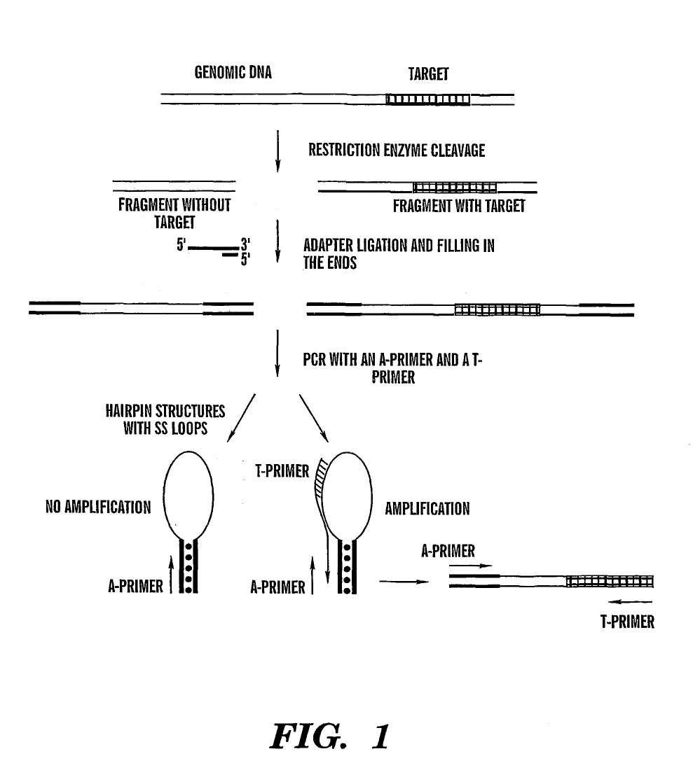

PS PCR relies on hairpin structures formed by all genomic fragments after DNA is digested with a restriction enzyme, ligated with long GC-rich PS adapters, denatured and re-annealed (FIG. 1). PS PCR usually uses primers that tolerate high primer-annealing temperatures (13) As a result, the specificity of PS PCR is extremely high (13, 17, 18).

The available experimental data suggest that PS PCR is well suited for multiplex PCR. Several issues, however, need to be addressed to optimize this method for multiplex amplification. To perform PS PCR, genomic DNA is digested with an appropriate restriction enzyme and ligated with PS-adapters. Thus the design of the gene-specific primers depends on the location of the target and also on the availability of a proximal restriction site. Another problem, which is inherent to all PCR-based methods with multiple targeting, is preferential amplification of certain templates and loss of others during repeated PCR cycles.

### Example 1

Protocol A: Preparation of Genomic DNA with Ligated Adapters.

Combine in a 1.5 ml sterile Eppendorf tube 5 μl (500 ng) of human genomic DNA: 5 μl of 10× New England Biolabs buffer 1, 2 μl of RsaI (10 Unit/μl, NE Biolabs) and add sterile water to 50 μl. Mix contents and spin the tubes briefly in a centrifuge.

1. Incubate at 37° C. for 90 min.

2. Add 1 μl of RsaI (10 Unit/μl, NE Biolabs)

3. Incubate at 37° C. for 90 min.

4. Purify the digest by using Qiaquick DNA purification kit (Qiagen) following the manufacturer protocol. Elute DNA fragments with 40 μl of hot sterile water.

5. In a separate 1.5 ml Eppendorf tube mix equal volumes of 40 μM solutions of a long and short adapters in a buffer containing 10 mM Tris-HCl, pH 7.5, 0.1 mM EDTA and 100 mM NaCl.

6. Incubate the adapter mixture at 70° C. for 15 min and let the mixture cool at ambient temperature for 20-30 min.

7. Mix the following reagents in the order shown:

| | | |
|---|---|---|
| Digested DNA | 40 | μl |
| 20 μM adapters | 4 | μl |
| 10x ligation buffer | 5 | μl |
| T4 DNA ligase 3 Unit/μl | 1 | μl |

8. Vortex and spin briefly in an Eppendorf microcentrifuge.

9. Incubate at ambient temperature overnight.

10. Incubate at 75° C. for 5 min to inactivate ligase.

11. Vortex and spin briefly in an Eppendorf microcentrifuge.

12. Purify ligate by using Qiaquick DNA purification kit (Qiagen) following the manufacturer protocol. Elute DNA fragments with 40 μl of hot sterile water.

Protocol B: Multiplex PCR with Multiple Sequence-specific Primers

1. For one PCR amplification (final volume 25 μl) mix the following reagents in 200 μl PCR tube:

| | | |
|---|---|---|
| 10x PCR buffer (10 mM tris-HCl, pH 8.3, 50 mM KCl) | 2.5 | μl |
| 25 mM MgCl₂ | 2.5 | μl |
| dNTPs, 2.5 mM each dATP, dCTP, dGTP, dCTP | 2.5 | μl |
| Adapter ligated DNA (3-5 ng) | 1 | μl |
| AmpliTaq DNA polymerase (5 Un/μl) | 0.5 | μl |
| Sterile water | 11 | μl |

2. In a separate 200 μl PCR tube mix 5 pmole of each primer (0.5 μl of 10 μM stock solution) and add sterile water to 5 μl. For example, to amplify 5 targets (Table 1), 6 primers are needed: 5 primers from Table 1 and a fluorescein-labeled adapter-primer TGTAGCGTGAAGACGACAGAA. SEQ ID NO.: 1)

3. If the number of primers exceeds 10, use higher concentration of the stock primer solution.

4. Place both tubes, one with the primers, another with all other components of the PCR, in a PCR cycler (MJ Research., Watertown, Mass.) and heat the block to 94° C.

5. Add primers to the PCR mixture at 94° C.

6. Immediately start 38 cycles program: 94° C., 10 sec, 68° C., 15 sec, 72° C., 1 min (as in Example 1).

7. Alternatively, use another cycling program: 2 cycles, 94° C., 10 sec, 65° C., 15 sec, 72° C., 1 min; 2 cycles, 94° C., 10 sec, 67° C., 15 sec, 72° C., 35 cycles, 94° C., 10 sec, 69° C., 15 sec, 72° C., 1 min (as in Example 2).

8. Analyze 5 μl of the PCR product on a 2% agarose gel, 1× TAE buffer and use 100 bp size marker (Gibco-BRL). 9. Alternatively, mix 2 μl of the PCR product with 4 μl of the loading dye (Pharmacia Biotech), denature at 94° C. for 1 min in a thermocycler and chill on ice.

10. Load 6 μl on a 6% sequencing polyacrylamide gel in 0.6× TBE buffer. Use fluorescent 50 bp size marker (Pharmacia Biotech).

11. Run the gel in the ALF automated sequencer. Use Fragment Manager software to analyze the data.

### Example 2

14-plex PCR Using Human Genomic DNA

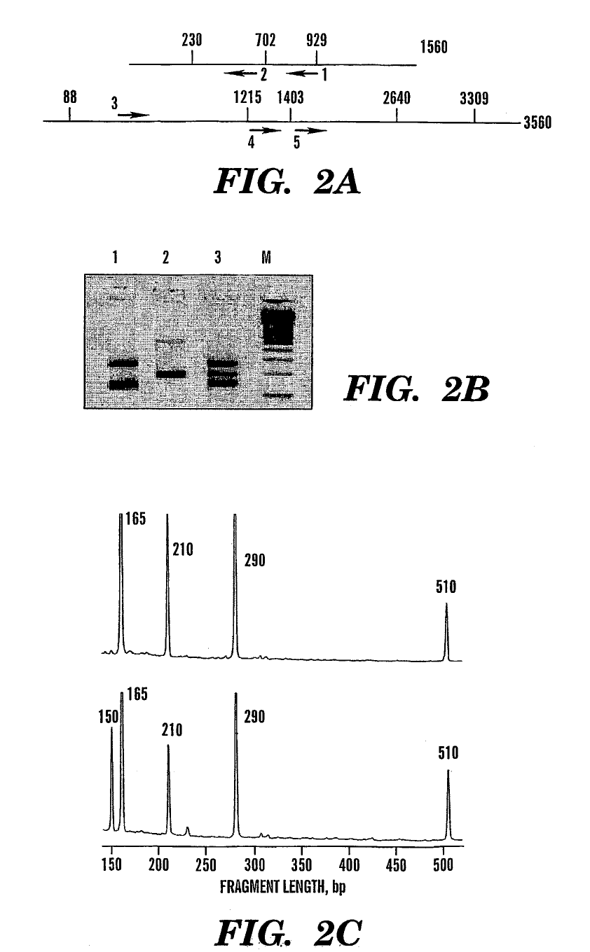

To demonstrate PS-based mpxPCR, we chose two fragments (1560 and 3560 base pairs) from a large sequenced segment on human chromosome 7q22 (accession number AF053356), and generated their RsaI maps. Five primers were chosen to amplify 5 corresponding targets (see FIG. 2A for the scheme and Table 1). Human genomic DNA from an anonymous blood donor was digested with RsaI restriction enzyme and ligated with PS-adapters. The PS-adapters differed from the adapter sequences in Chenchik et al. patent in that they contained a build-in type IIS restriction site, which will be used in our further manipulations; namely SEQ ID NO: 22 and 23.

| | |
|---|---|
| 5′TGTAGCGTGAAGACGACAGAA AGGGCGTGGTGC | (SEQ ID NO:22) |
| GGACGCGGGT3′ | |
| 3′CGCCTGCGCCCA5′ | (SEQ ID NO:23) |

The specificity of all primers was first tested in single reactions using the A-primer and the corresponding T-primer. In all but one case (primer 5, Table 1), single products of the expected sizes were generated. Primer 5 generated one additional fragment of smaller than expected size (Table 1). The nature of this product was not studied. Next, PCRs with different multiplexing using equimolar mixtures of T-primers and the A-primer were performed. In these experiments, the A-primer was fluorescently labeled, and 35-38 cycles of PCR were performed to visualize products from 2-5 ng of genomic DNA.

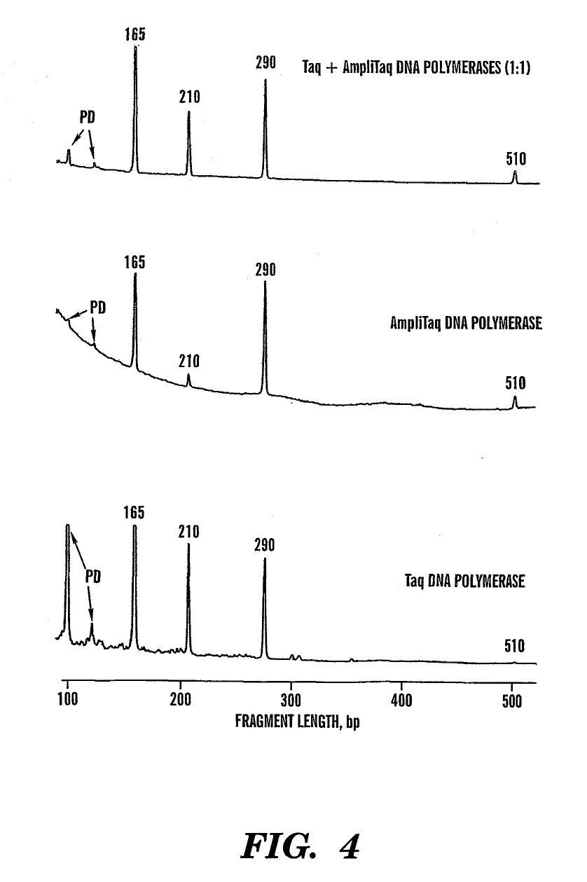

FIG. 2B shows the products of 2-plex and 4-plex PCRs after agarose gel electrophoresis and FIG. 2C presents 4-plex and 5-plex PCR products after resolution on polyacrylamide gels. All targeted amplicons were detected as PCR products, and the amounts of amplicons in the range 100-400 base pairs were similar. However, a ˜500 bp amplicon was synthesized in smaller amounts (see also FIG. 4 and below).

TABLE 1

PCR target specific primers targeted at several
anonymous sequences in chromosome 7 used in the
multiplex PCR.

| | |
|---|---|
| Direction, | |
| length (nt), | Product length (bp) |

| | | | |
|---|---|---|---|
| Primer (5′-3′) | GC content | Expected | Obtained |

| | | | |
|---|---|---|---|
| 1. aatgcctgccatgtataagctacccggtc | Reverse, 29, | 165 | 165 |
| (SEQ ID NO:2) | | | |
| 2. gtcccgtccccatcctcacaagctgtcgc | Reverse, 29, | 285 | 290 |
| (SEQ ID NO:3) | 19/29 | | |
| 3. agtgcccatgcccgtgagacctggagaag | Direct, 29, | 507 | 510 |
| (SEQ ID NO:4) | 18/29 | | |
| 4. ccggaggaaattggagtagactcggaagag | Direct, 30, | 212 | 210 |
| (SEQ ID NO:5) | 16/30 | | |
| 5. gcagccccaagcaccaagctgagcaaacag | Direct, 30, | 970 | ˜1000 |
| (SEQ ID NO:6) | 18/30 | | 150 |

### Example 3

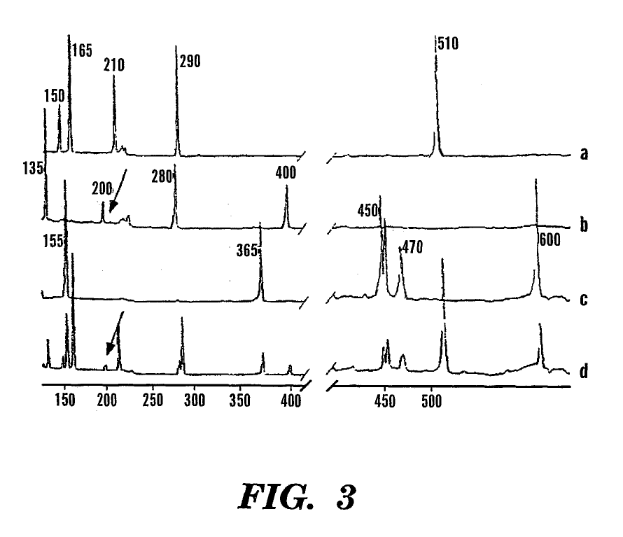

In a different experiment, we chose to amplify portions of 12 genes known to have single base variations at biallelic loci (9). The primers were taken from Belgrader et al. (1998) and extended by several bases (for different primers extension was from different sides or from both sides), so that all gene-specific primers were 30-33 bases long, depending on the GC content. The choice between the direct or reverse primer was determined by the location of the nearest RsaI site. All 12 primers were tested in single reactions, and three primers (cytochrome 450IID gene, C6 complement gene and S-beta pseudogene) were excluded from our subsequent experiments because of inadequate specificity. The primer for the aldolase B gene (primer 4, Table 2) generated a PCR product, which appeared as a double peak on a polyacrylamide gel. Multiplex PCR with different combinations of gene-specific primers, including a mixture of all nine T-primers, generated bands with sizes that corresponded to the expected ones. FIGS. 3a, b and c show the patterns obtained in 5-plex PCR (primers 1-5, Table 1), 4-plex PCR (primers 2, 3, 5 and 8, Table 2) and 5-plex PCR (primers 1, 4, 6, 7, and 9, Table 2). Finally, we performed a 14-plex PCR, in which we combined the primers for the 5 fragments from chromosome 7 (Table 1) and 9 gene-specific primers from Table 2 (primers 1-9). In this experiment all 14 targeted amplicons were detected (FIG. 3D). The relative amounts of the amplicons inversely correlated with the size of the fragments and also depended of the annealing temperature (Tm) of the T-primer (Table 3, see also below). For example, the relatively low concentration of the interleukin 1-alpha amplicon (FIG. 3, marked by arrow) correlated with the lowest Tm of the corresponding primer.

TABLE 2

PCR primers targeted at bialleleic loci and the CFTR locus
used in PS mpxPCR. Primers targeted at the interleukin 1
alpha gene, neurofibromatosis 1 locus and
alpha₂-macroglobulin were used as a normal (n) and a double
mismatch (m) variant. ND—not determined.

| | |
|---|---|
| Direction, | |
| length (nt), | Product length (bp) |

| | | | |
|---|---|---|---|
| Primer (5′-3′) | GC content | Expect | Obtain |

| | | | |
|---|---|---|---|
| 1. Antithrombin III gene | Reverse, 30, | 604 | ˜600 |
| GGTCCCATC TCCTCTACCTGATACAGACTC | 16/30 | | |
| (SEQ ID NO:7) | | | |
| 2. Interleukin 1 alpha (IL-1 alpha) gene | Direct, 32, | 197 | 200 |
| n CTGCAC TTG TGATCATGG TT T TAGAAATCATC | 12/32 | | |
| (SEQ ID NO:8) | | | |
| m CTGCACTTG TGATCATGG TT T TAGAAATAA TA | | | |
| (SEQ ID NO:9) | | | |
| 3. Neurofibromatosis 1 locus | Reverse, 31, | 276 | 280 |
| n GAGGACCATGGCTGAGTCTCCTTTAGTGTCC | 17/31 | | |
| (SEQ ID NO:10) | | | |
| m GAGGACCATGGCTGAGTCTCCTTTAGTATC A | | | |
| (SEQ ID NO:11) | | | |
| 4. Aldolase B gene, | Direct, 30, | 449 | 450* |
| GGCTTGACTTTCCAACACGGAGAAGCATTG | 15/30 | | |
| (SEQ ID NO:12) | | | |
| 5. Alpha₂-macroglobulin gene | Reverse, 33, | 135 | 135 |
| n CCCTTACTCAAG TAATCACTCACCAGTG TTGAG | 15/33 | | |
| (SEQ ID NO:13) | | | |
| m CCCTTACTC AAG TAATCACTCACCAGTG TAGAA | | | |
| (SEQ ID NO:14) | | | |
| 6. Insulin-like growth factor II gene | Reverse, 32, | 468 | 470 |
| ACCCTGAAAATTCCCGTGAGAAGGGAGATGGC | 17/32 | | |
| (SEQ ID NO:15) | | | |
| 7.Triglyceride lipase gene, exon 4 | Direct, 30, | 155 | 155 |
| CAACACACTGGACCGCAAAAGGCTTTCATC | 15/30 | | |
| (SEQ ID NO:16) | | | |
| 8. Integrin B₂ subunit gene | Reverse, 30, | 400 | 400 |
| CGGGCGCTGGGCTTCACGGACATAGTGACC | 20/30 | | |
| (SEQ ID NO:17) | | | |
| 9. Low-density lipoprotein receptor gene | Reverse, 30, | 365 | 365 |
| CAGAGACAGTGCCCAGGACAGAGTCGGTCC | 19/30 | | |
| (SEQ ID NO:18) | | | |
| 10. Cystic fibrosis trausmembrane | Reverse, 40, | 355 | 350 |
| regulator gene, (n) | | | |
| GACGCTTCTGTATCTATATTCATCATAGGAAACACCA | 15/40 | | |
| AAG | | | |
| (SEQ ID NO:19) | | | |
| 11. Cystic fibrosis transmembrane regulator | Reverse, 39, | 355 | 350 |
| gene, ΔF508, | 14/39 | | |
| GACGCTTCTGTATCTATATTCATCATAGGAAACACCA | | | |
| AT | | | |
| (SEQ ID NO:20) | | | |
| 12. Cystic fibrosis transmembrane regulator | Reverse, 29 | 372 | ND |
| gene, nested | 13/29 | | |
| TCTTCTAGTTGGCATGCTTTGATGACGCT | | | |
| (SEQ ID NO:21) | | | |

*This product appeared as a double peak (see FIG. 3)

### Example 4

Different DNA Polymerases Display Different Specificity in mpxPCR

Three parameters are important to evaluate the quality of multiplex amplification reaction: (i) the uniformity of amplification of different targets, (ii) the amount of primer-dimers, and (iii) the non-specific background or signal to background ratio. FIG. 4 presents the results obtained with two different DNA polymerases, AmpliTaq and Taq DNA polymerase, in an experiment designed to amplify four amplicons (Table 1, primers 1-4). The data show that Taq DNA polymerase amplifies 100-400 bp amplicons more uniformly than AmpliTaq; however, this is accompanied by a greater amount of primer-dimers (FIG. 4). In addition, the 510 bp fragment is amplified by Taq DNA polymerase much less efficiently than by AmpliTaq. At the same time, AmpliTaq DNA polymerase generates much higher non-specific background and amplifies 100-400 bp amplicons less uniformly that Taq DNA polymerase. To keep a low background and low primer-dimers and still amplify all the targets, we used a 1.1 mixture of both polymerases, which produced the best result (FIG. 4). We also tested KlenTaq and AmpliTaq Gold DNA polymerases and found that the combination of these DNA polymerases with AmpliTaq or Taq DNA polymerase also generates a good amplification pattern with low background (data not shown). At the moment we typically use a mixture (1:1:1) of three different DNA polymerases, AmpliTaq, AmpliTaq Gold and Taq DNA polymerases.

### Example 5

Allele-specific mpxPCR

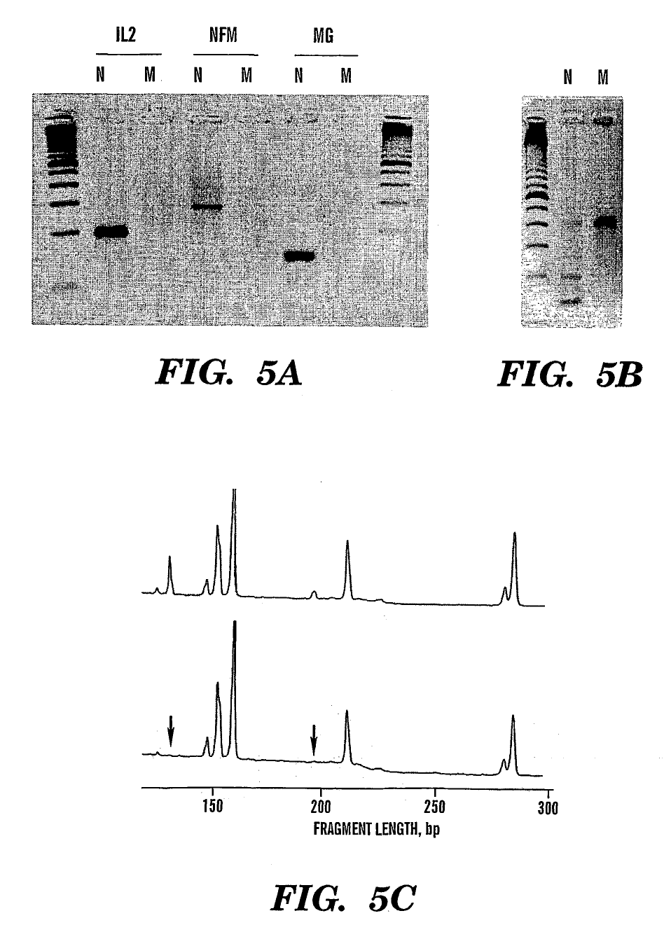

To be useful for mutation detection the PCR should discriminate between alleles, and single-base discrimination should be routinely attainable. We performed mpxPCR with primers containing a 3′-terminal mismatched base, imitating allele-specificity. In addition, a penultimate base was mismatched to increase the impact of the 3′-mismatch (20-22). FIG. 5a shows that uniplex PS PCR with such mismatched primers targeting interleukin, neurofibromatosis and alpha-2-macroglobulin genes perfectly discriminated against a 3′-end mismatch nucleotide. Next, we performed a 4-plex PCR with a mixture of these three primers, either wild or mutated, together with a fourth primer, which was the same in both wild and mutant reactions (it was targeted at an integrin B₂ subunit gene fragment, primer 8, Table 2). FIG. 5b shows an agarose gel where three bands, corresponding to interleukin, neurofibromatosis and alpha-2-macroglobulin gene fragments are essentially absent, when the corresponding mismatched primers were used for amplification. In addition, FIG. 5c shows the absence of the interleukin and macroglobulin gene fragments amplified in a 14-plex PCR with the corresponding mutated primers. These data suggest that this method can be used for multiplex genotyping of DNA samples.

### Example 6

Genotyping of DNA Samples from Cystic Fibrosis-affected Individuals Using the PS based mpxPCR

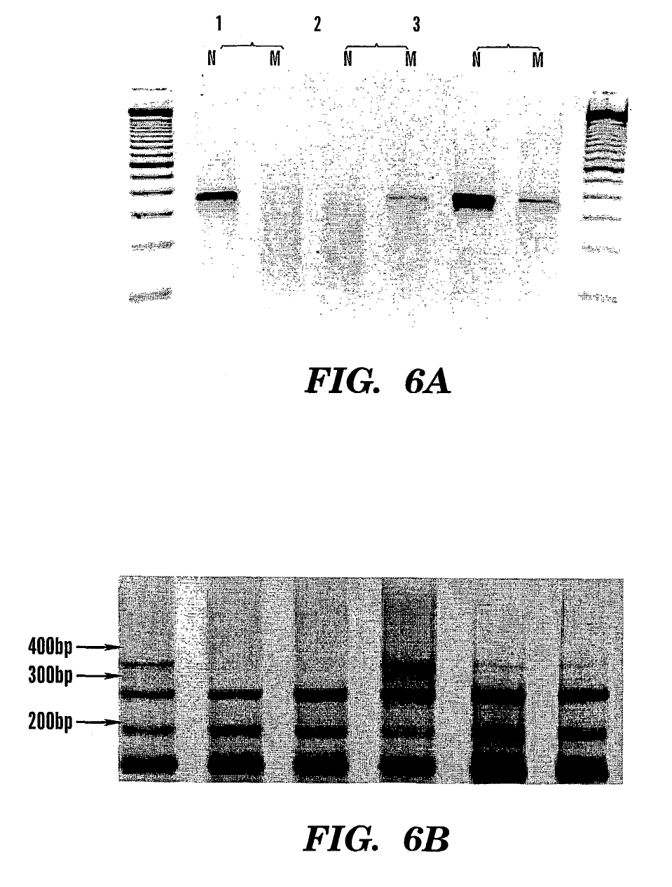

The applicability of mpx PS PCR in disease diagnostics was shown by genotyping of DNA samples from CF-affected individuals with the ΔF508 mutation consisting in a deletion of three nucleotides in exon 10 of the CFTR gene (23). The region around the ΔF508 mutation is extremely AT-rich; therefore 39-40 base-long reverse CFTR-primers were designed to survive the 68° C. annealing temperature (Table 2). The choice of the reverse primer was determined by the location of an RsaI site about 300 bp upstream of the ΔF508 mutation point. The normal and mutated primers were designed to differ by one nucleotide in length and also by the 3′-terminal nucleotide (Table 2). However, preliminary experiments showed that neither primer provided adequate specificity. Therefore, in this particular case we performed nested PCR and used in the first step a 29-mer nested CFTR primer (primer 12, Table 2) in combination with the 5′-outmost adapter-primer in a 15 cycle PCR. Then 1 μl of the PCR product was diluted 500-fold and re-amplified in a 38 cycle PCR with either normal or mutant CFTR-primers (primers 10 or 11, respectively) and 3′ A-primer. FIG. 6A shows that only the normal CFTR-primer amplifies an expected 350-bp fragment from unaffected DNA; both primers generate products from CFTR-heterozygous DNA; and only a mutant primer amplifies a product from ΔF508 CFTR-homozygous DNA. The lower efficiency of PCR with the CFTR mutant primer (FIG. 6A) is due to the shorter length and, consequently, the lower Tm of the corresponding primer (see Table 2). It is remarkable, however, that the difference between the normal and mutated CFTR primers is practically eliminated in mpxPCR (see below and FIG. 6B).

30-plex PCR

In another example, mpx PCR was used to amplify 30 target sequences, 14 of the target sequences and the target primers were those used for the 14-plex PCR described above (Example 2); 16 additional target sequences and target primers are shown in Table 3. For multiplex PCR of 30 targets, the extension time of the PCR reaction was tripled, to 3 minutes. Finally, the concentration of primers was increased, particularly for those targets underrepresented in the final reaction product. Our results indicate that the multiplex PCR method of the present invention can detect 30 different sequences amplified in a single reaction.

TABLE 3
Primers used to amplify SNPs

| | | | | |
|---|---|---|---|---|
| | | | Tm | |
| | | (nt) | product | |
| # SNP name | primer sequence (5′-3′) | length | (bp) | length |

| | | | | | |
|---|---|---|---|---|---|
| 1. WIAF-65 | gacacatgga ggcttagttc agggctttgg gcc | SEQ ID NO:31 | 33 | 70.3 | 230 |
| 2. WIAF-936 | gagtgaagaa tgggcctcat gtcacacgag g | SEQ ID NO:32 | 31 | 67.1 | 320 |
| 3. WIAF-1556 | ggagttaagc tatgggtatg caaaggcata c | SEQ ID NO:33 | 31 | 62.7 | 130 |
| 4. WIAF-163 | gatagctcct gagacactgg ccctgtctag g | SEQ ID NO:34 | 31 | 67.3 | 325 |
| 5. WIAF-243 | tggcaggggt gggaggtcag actttcccta gag | SEQ ID NO:35 | 33 | 71.4 | 290 |
| 6. WIAF-1841 | gcttcattca acaatgagcc tcacagccgt gc | SEQ ID NO:36 | 32 | 68.4 | 87 |
| 7. WIAF-2012 | cactgtagag aaaagtgaag tataaaatgg ggtc | SEQ ID NO:37 | 34 | 60.7 | 530 |
| 8. WIAF-963 | catagagatt tgagttttca cctaggtttt ctcc | SEQ ID NO:38 | 34 | 60.8 | 92 |
| 9. WIAF-1797 | gataatagtg tccacctgat cacccagatc agcc | SEQ ID NO:39 | 34 | 65.9 | 160 |
| 10. WIAF-284 | gaaagaagtc ctcttcaatc ccttatcctg gag | SEQ ID NO:40 | 33 | 63.1 | 280 |
| 11. WIAF-925 | ctgtgtgaac tcgaattcgc ttgtccagtc ctg | SEQ ID NO:41 | 33 | 67.0 | 170 |
| 12. WIAF-756 | cctaacagcc ttggaaggca ggtaaactgt tgc | SEQ ID NO:42 | 33 | 67.5 | 560 |
| 13. WIAF-185 | gtgaaagatg gaaacgagtt ttcacatgtg | SEQ ID NO:43 | 30 | 60.6 | 175 |
| 14. WIAF-567 | gaggaatcat gctggggcaa ggattgcagt tgaag | SEQ ID NO:44 | 35 | 68.1 | 330 |
| 15. WIAF-908 | gagagaggtg aaatgacttg ctcaagccga gtc | SEQ ID NO:45 | 33 | 66.6 | 150 |
| 16. WIAF-1653 | catcttcctt ctgccagtta aacgtgccgt ggc | SEQ ID NO:46 | 33 | 69.1 | 78 |

Multiplex allele-specific PCR was shown by genotyping of the CFTR locus on the background of 3 other loci (primers 1, 2, and 4, Table 1) in five DNA samples, which supposedly contained three homozygous and two heterozygous ΔF508 CFTR mutations. In these experiments, we performed two-step mpxPCR as described above. FIG. 6B exemplifies the results. ΔF508 CFTR homozygous DNA samples were positive with the mutant CFTR primer and negative with the normal CFTR primer (FIG. 6B). Heterozygous ΔF508 CFTR samples showed the presence of the CFTR fragment with both, normal and mutant CFTR primers, thus, confirming ΔF508 heterozygosity. In all DNAs tested by multiplex genotyping, the status of CFTR mutation was identified correctly and was confirmed by direct sequencing.

This data shows the value of the present invention comprising mpxPCR utilizing the PS-effect (13, 24). This method requires about half the number of primers as compared to conventional mpxPCR. This substantially simplifies primer design and brings down primer costs. Primer cost savings are typically at least 1.5 fold. For example, in conventional PCR aimed to amplify 12 biallelic loci (9) the average length of the sum of the two gene-specific primers was 45 (38-62) nt, while in the PS PCR the same targets were amplified with an average primer that was 31 (30-33) nt long (see Table 2, primers 1-9), and only a single labeled primer was required.

Using this method, we have performed 30-plex, 14-plex and other sized PCR targeting DNA fragments from various human chromosomes. It is important to emphasize that all T-primers efficient in single reactions were efficient also in mpxPCR, and none of the T-primers was found to be non-compatible with others. Therefore, this approach can be adapted for various kinds of studies. As the number of targets increases, minor adaptations such as increasing the length of extension times should be made.

The preparation of DNA samples for PS PCR includes digestion of genomic DNA with an appropriate restriction enzyme and ligation with the PS-driving adapters. These extra-steps, however, are not burdensome since the DNA samples prepared can be used in multiple experiments. It is preferably to choose one restriction enzyme for as many of the targets as possible. Preferably, all targets. In most of these examples, targets were not dropped because of the lack of a RsaI site in the range accessible to amplification. Therefore, we believe that the choice of a single restriction enzyme for most of the targets should be not a problem, and a suitable site will be available in proximity to practically every target.

The PCR conditions, which we used for mpxPCR, did not require special optimization and should be considered as default conditions applicable for amplification of any target, provided the primer's Tm tolerates 65-68° C. We have found that the PCR cycling conditions have little impact on the amplification result (data not shown). The hairpin-loop structures acquired by all genomic fragments we believe present a positive factor for efficient target amplification. Indeed, all the targets are located within the ss regions of the hairpin-loop structures and are, therefore, available to corresponding primers for binding. This may explain the fact that the relative amplification efficiency of similar sized targets in mpxPCR correlates with the Tm of the primers (Table 4). The size of the ss loop seems to be another factor affecting the efficiency of PS PCR. A target residing in a larger ss loop (˜600 nt) is amplified less efficiently than a target located in a smaller ss loop (227 nt) (see Table 4). This may occur because larger ss loops form stable secondary structures more easily than shorter ones; this can inhibit annealing of the corresponding T-primer and decrease the yield of the amplicon. The size of the ss loop is determined by the distance between the two restriction sites and can not be regulated by experimentator. However, the decreased efficiency of amplification of targets with large ss loops can be partly compensated by increasing the length of the primer and consequently its Tm.

TABLE 4
Dependence of the amplicon yield on the Tm of the primer in the
multiplex PS PCR. The Tm's of the primers were calculated
using a nearest-neighbor thermodynamic parameter set (19, see
also http://www.idtdna.com/technotes_facs/Calculating_Tm).
Peak areas were determined by analyzing a 14-plex PCR
pattern using Fragment manager software provided with
the ALF sequencing instrument.

| | | Product | | |
|---|---|---|---|---|
| | | size, | ss loop | Peak area, |
| Primer | Tm, ° C. | bp | size, nt | arbitrary units |
| #2, Table 2 | 60.40 | 200 | 170 | 310 |
| #5, Table 2 | 63.36 | 135 | 308 | 1020 |
| #4, Table 1 | 64.70 | 210 | 188 | 2180 |
| #7, Table 2 | 65.69 | 155 | 607 | 2590 |
| #l, Table 1 | 65.72 | 165 | 227 | 4630 |

The general trend of decreased PCR amplification efficiency for longer targets is seen also in mpx PS PCR. The need of different size targets is determined solely by the use of gel-based methods for resolution and analysis of the amplification products. If the analysis procedure avoids gel-based separation methods (e. g. DNA microarray-based analysis of the PCR products (25) or mass spectrometry (26)), it will be possible to design primers to generate PCR products of very similar size. This should help in more uniform amplification of different targets and might further increase the level of achievable multiplexing.

Different thermostable DNA polymerases displayed different specificity in PS mpxPCR. Mixtures of DNA polymerases generated more uniform amplification patterns than individual DNA polymerases. (FIG. 4). We speculate that minor differences in strand displacement activity and processivity of different DNA polymerases may cause differences in specificity. Mixes of DNA polymerases have been already used in PCR for other purposes. For example, a combination of two thermostable DNA polymerases, one of which had 3′-5′ proofreading activity, has been shown to increase the length of PCR amplification (27).

In PS mpxPCR, the criteria for primer selection are, in general, the same as in conventional mpxPCR: the primers should not be self- or mutually complementary and should not form homo- and hetero-dimers (28). Because PS PCR requires only one specific primer, its uniqueness is crucial. Therefore, the primers are usually relatively long oligonucleotides with high GC contents. This allows high annealing temperatures and thereby increases the specificity (13). In order to increase the specificity further, one can perform nested PCR (13, 18, 24). For the purposes of multiplexing, however, nested PCR may not be required. Our data showed that 30 to 32-base-long target-primers combined with the 5′-outmost adapter-primer provide specific amplification for most of the targets (see Table 1 and 2), and, therefore, nested primers are not obligatory for successful multiplexing. During this study, the only one target was encountered, which was not amplified with 30 to 32-base-long primers (CFTR gene). The region around the ΔF508 mutation is extremely AT-rich, and to reach the Tm's of 67-68° C. it was necessary to use 40-base-long primers and perform nested PCR from the target side. However, in all other cases we successfully generated PCR amplicons by performing one-step PCR. This substantially simplified the protocol compared to two-step procedures.

The necessity of only one gene-specific primer adds much flexibility to primer design and allows the use of primers complementary to either of the two DNA strands depending on the availability of the closest restriction site. This is especially important for amplification of homologous gene-family members or repetitive sequences, where it is often difficult to choose two distinct specific primers for each gene-family member.

The extremely high specificity of PS PCR allows allele-specific amplification with single-base discrimination. In our experiments we tested G/A and C/A mismatches which display moderate destabilizing effects (29) and correspond to common C-T and G-T variations (30). The fact that allele-specificity has been attained through 30-plex PCR, demonstrates the exquisite fidelity of this approach. Another advantage of this technique consists in its amenability to automation and development of high-throughput genetic diagnostics. For example, by performing 14-plex PCR in 96-wells microtitre plate, one will be able to analyze 192 chromosomes at 14 loci simultaneously. This method can be easily combined with various advanced techniques, e. g., two-color detection and microarray-based analysis of the PCR products, which will increase further the throughput and information content of every experiment.

References

1. Chamberlain, J. S., Gibbs, R. A., Ranier, J. E., Nguyen, P. N., & Caskey, C. T. (1988) Nucleic Acids Res 16, 11141-11156.

2. Edwards, M.C., &. Gibbs R. A. (1994) PCR Methods Appl 3, S65-75.

3. Hacia, J. G., Sun, B., Hunt, N., Edgemon, K., Mosbrook, D., Robbins, C., Fodor, S. P., Tagle, D. A., & Collins F. S. (1998) Genome Res 8, 1245-1258.

4. Li, D., & Vijg, J. (1996) Nucleic Acids Res 24,538-539.

5. Stuven, T., Griese, E. U., Kroemer, H. K., Eichelbaum, M. & Zanger, U. M. (1996) Pharmacogenetics 6, 417-421.

6. van Orsouw, N. J., Zhang, X., Wei, J. Y., Johns, D. R., & Vijg, J. (1998) Genomics 52, 27-36.

7. Rines, R. D., van Orsouw, N. J., Sigalas, I., Li, F. P., Eng C., & Vijg, J. (1998) Carcinogenesis 19, 979-984.

8. Shuber, A. P., Grondin, V. J., & Klinger, K. W. (1995) Genome Res 5,488-493.

9. Belgrader, P., Marino, M. M., Lubin, M., & Barany, F. (1996) Genome Science & Technology 1, 77-87.

10. Brownie, J., Shawcross, S., Theaker, J., Whitcombe, D., Ferrie, R., Newton, C., & Little S. (1997) Nucleic Acids Res 25, 3235-3241.

11. Hacia, J. G., Sun, B., Hunt, N., Edgemon, K., Mosbrook, D., Robbins, C., Fodor, S. P., Tagle, D. A., & Collins, F. S. (1998) Genome Res 8,1245-1258.

12. Launer, G. A., Lukyanov, K. A., Tarabykin, V. S., &. Lukyanov, S. A. (1994) Mol Gen Mikrobiol Virusol 6, 38-41.

13. Siebert, P. D., Chenchik, A., Kellogg, D. E., Lukyanov, K. A., & Lukyanov, S. A. (1995) Nucleic Acids Res 23, 1087-1088.

14. Diatchenko, L., Lau, Y. F., Campbell, A. P., Chenchik, A., Moqadam, F., Huang, B., Lukyanov, S., Lukyanov, K., Gurskaya, N., Sverdlov, E. D., & Siebert, P. D. (1996) Proc Natl Acad Sci U S A 93, 6025-6030.

15. Akopyants, N. S., Fradkov, A., Diatchenko, L., Hill, J. E., Siebert, P. D., Lukyanov, S. A., Sverdlov, E. D., & Berg, D. E. (1998) Proc Natl Acad Sci USA 95, 13108-13113.

16. Diatchenko, L., Lukyanov, S., Lau, Y. F., & Siebert, P. D. (1999) Methods Enzymol 303, 349-380.

17. Broude, N. E., Storm, N., Malpel, S., Graber, J. H., Lukyanov, S., Sverdlov, E., & Smith, C. L. (1999) Genet Anal 15, 51-63.

18. Lavrentieva, I., Broude, N. E., Lebedev, Y., Gottesman, I. I., Lukyanov, S. A., Smith, C. L., & Sverdlov, E. D. (1999) FEBS Lett 443, 341-347.

19. Allawi, H. T., & SantaLucia, J. Jr. (1997) Biochemistry 36, 10581-10594.

20. Norby, S., Lestienne, P., Nelson, I., & Rosenberg, T. (1991) Biochem Biophys Res Commun 175, 631-636.

21. Cha, R. S., Zarbl, H., Keohavong, P., & Thilly, W. G. (1992) PCR Methods Appl 2,14-20.

22. Matsunaga, J., Tomita, Y., & Tagami, H. (1995) Exp Dermatol 4, 377-381.

23. Zielenski, J., Rozmahel, R., Bozon, D., Kerem, B., Grzelczak, Z., Riordan, J. R., Rommens, J., & Tsui, L. C. (1991) Genomics 10, 214-28.

24. Lukyanov, K. A., Launer, G. A., Tarabykin, V. S., Zaraisky, A. G., & Lukyanov, S. A. (1995) Anal Biochem 229, 198-202.

25. Gerry, N. P., Witowski, N. E., Day, J., Hammer, R. P., Barany, G., & Barany, F. (1999) J Mol Biol 292, 251-62.

26. Tang, K., Fu, D. -J., Julien, D., Braun, A., Cantor, C. R., & Koester, H. (1999) Proc Natl Acad Sci U S A 96, 10016-10020.

27. Barnes, W. M. (1994) Proc Natl Acad Sci U S A 91: 2216-2220.

28. Rychlik, W. (1995) Mol. Biotechnol. 3, 129-134.

29. Cotton, R. G. H. (1989) Biochem J 263, 1-10.

30. Cargill, M., Altshuler, D., Ireland, Sklar, J., Ardlie, P. K., Patil, N., Shaw, N., Lane, C. R., Lim, E. P., Kalyanaraman, N., et al. (1999) Nat Genet 22, 231-238.

All patent applications, patents, and literature references cited in this specification are hereby incorporated by reference in their entirety.

What is claimed is:

1. A method for simultaneously detecting the presence of multiple target DNA sequences in a population of nucleic acid template molecules, wherein each member of said population of nucleic acid template molecules has a tennis-racquet shaped structure comprising a double-stranded handle region and a single-stranded head region, wherein the target gene of interest lies within the single-stranded head region and has been generated by attaching a PCR suppression adapter to each end of a nucleic acid fragment in a nucleic acid sample, such that the adapter sequence falls within the double-stranded handle region, comprising:
(a) contacting said population of nucleic acid template molecules with a multiplicity of single-stranded oligonucleotide DNA primers, wherein said multiplicity of single-stranded oligonucleotide DNA primers comprises a universal adapter oligonucleotide DNA primer complementary to a region within said adapter sequence and at least five different target-specific oligonucleotide DNA primers, wherein each of said target-specific oligonucleotide DNA primers comprises a nucleotide sequence that is complementary to a nucleotide sequence specific for a target nucleic acid fragment and is designed to fall within the single-stranded head region, whereby the synthesis of spurious amplification products is prevented;
(b) adding to said mixture obtained after step (a) an effective amount of reagents necessary for performing a polymerase chain reaction;
(c) cycling the mixture obtained after step (b) through at least one cycle of the denaturing, annealing and primer extension steps of PCR to form amplification products for each of said multiple target DNA sequences amplified with said multiplicity of single-stranded oligonucleotide DNA primers; and
(d) detecting said amplification products.

2. The method of claim 1, wherein detection of said amplification products indicates the presence of said multiple target DNA sequences in said population of nucleic acid template molecules.

3. The method of claim 1, wherein said PCR reagents comprise at least one thermostable DNA polymerase selected from the group consisting of Taq, Ampli Taq, Ampli Taq Gold, Tth, Pfu and Vent.

4. The method of claim 1, wherein said PCR reagents comprise a combination of at least three DNA polymerases selected from the group consisting of Taq, Ampli Taq, Ampli Taq Gold, Tth, Pfu and Vent.

5. The method of claim 1, wherein said population of nucleic acid template molecules comprises nucleic acid selected from the group consisting of genomic DNA, cDNA and RNA.

6. The method of claim 1 wherein at least 10 different target-specific oligonucleotide primers are present.

7. The method of claim 1, wherein at least 14 different target-specific oligonucleotide primers are present.

8. The method of claim 1, wherein said step of detecting comprises gel electrophoresis.

9. A mixture comprising a multiplicity of single-stranded oligonucleotide DNA primers for simultaneous amplification of multiple target DNA sequences under a single set of reaction conditions in a single multiplex polymerase chain reaction (PCR), wherein said multiplicity of single-stranded oligonucleotide DNA primers comprises a universal adapter oligonucleotide DNA primer and at least five different target-specific oligonucleotide DNA primers, wherein said primers are for use with tennis racquet-shaped nucleic acid template molecules, wherein said tennis racquet-shaped nucleic acid templates comprise a double-stranded handle region and a single-stranded head region, wherein a target sequence of interest lies within the single-stranded head region, and a repeating GC-rich sequence lies within said double-stranded handle region wherein said universal adapter oligonucleotide DNA primer comprises a nucleotide sequence that is complementary to a nucleotide sequence present, and each of said target-specific oligonucleotide DNA primer comprises a nucleotide sequence that is complementary to a nucleotide sequence specific for a target nucleic acid fragment within a single-stranded head region.

10. The method of claim 7, wherein said detecting step is carried out by high-throughput screening.

11. The method of claim 10, wherein said high throughput screening comprises DNA microarray screening.

12. The method of claim 11, wherein the PCR suppression adapter is a nucleotide sequence of 30 to 50 nucleotides, containing a restriction enzyme recognition site, and wherein said adapter contains at least 50% G or C nucleotides, and wherein said nucleotide sequence has less than 35% homology with said nucleic acid template molecules.

13. A method for simultaneously detecting the presence of multiple target DNA sequences in a DNA sample, comprising the steps of:
(a) attaching a PCR suppression adapter to each end of a nucleic acid fragment in said mixture;
(b) contacting said nucleic acid fragments having said attached adapters, in a single reaction mixture, with a multiplicity of single-stranded oligonucleotide DNA primers, wherein said multiplicity of single-stranded oligonucleotide DNA primers comprises a universal adapter oligonucleotide DNA primer, wherein said sequence comprises a nucleotide sequence that is complementary to a nucleotide sequence of said adapter and at least five different target-specific oligonucleotide DNA primers, wherein each target-specific oligonucleotide DNA primer comprises a nucleotide sequence that is complementary to a nucleotide sequence specific for a target nucleic acid fragment;
(c) adding to said mixture obtained after step (b) an effective amount of reagents necessary for performing a PCR;
(d) cycling the mixture obtained after step (c) through at least five cycles of the denaturing, annealing and primer extension steps of PCR to form amplification products for each of said multiple target DNA sequences amplified with said multiplicity of single-stranded oligonucleotide DNA primers; and
(e) detecting said amplification products.
